SEQUENCE LISTING (9 sequences)

SEQ ID NO: 1 - DNA, 29 bp, artificial
  8f primer
cacggatccagagtttgatnntggctcag

SEQ ID NO: 2 - DNA, 17 bp, artificial
  338r primer
gctgcctcccgtaggag

SEQ ID NO: 3 - DNA, 17 bp, artificial
  338f primer
ctcctacgggaggcagc

SEQ ID NO: 4 - DNA, 24 bp, artificial
  515f primer
tgccagcagccgcggtaatacgat

SEQ ID NO: 5 - DNA, 24 bp, artificial
  515r primer
atcgtattaccgcggctgctggca

SEQ ID NO: 6 - DNA, 17 bp, artificial
  968f primer
aacgcgaagaaccttac

SEQ ID NO: 7 - DNA, 17 bp, artificial
  968r primer
gtaaggttcttcgcgtt

SEQ ID NO: 8 - DNA, 17 bp, artificial
  1401r primer
cggtgtgtacaagaccc

SEQ ID NO: 9 - DNA, 31 bp, artificial
  1501r primer
gtcaagcttacggcttaccttgttacgactt

(19) United States
(12) Patent Application Publication
(10) Pub. No.: US 2009/0257993 A1
(43) Pub. Date: Oct. 15, 2009

(54) Lactic Acid Producing Bacteria and Lung Function

(76) Inventors: Laura M'Rabet, Amersfoort (NL); Gelske Speelmans, Wageningen (NL); Adrianus Johannes Maria Vriesema, Houten (NL); Johan Garssen, Nieuwegein (NL); Jan Knol, Wageningen (NL)

Correspondence Address: Jay S Cinamon;Abelman, Frayne and Schwab, 666 Third Avenue, New York, NY, 10017-5621, US

(21) Appl. No.: 10/583,171

(22) PCT Filed: Dec. 16, 2004
(86) PCT No.: PCT/NL04/00874

§ 371 Date: Apr. 29, 2009

(30) Foreign Application Priority Data
Dec. 17, 2003 (EP) 03079023.2

(51) Int. Cl. A61K 35/74; A61P 11/00; C12N 1/20

(52) U.S. Cl. 424/93.45; 424/93.4; 435/252.9; 435/252.1; 424/780

(57) ABSTRACT

The present invention provides novel use for live (probiotic) lactic acid producing bacteria, dead or non-viable bacteria thereof, as well as food supplements, nutritive compositions and/or pharmaceutical compositions comprising these, for the treatment or prophylaxis of lung dysfunction in a subject. A suitable lactic acid producing bacterium has a significant beneficial effect on airway narrowing determined by measuring the enhanced pause value (Penti) of a test animal.

### FIELD OF THE INVENTION

The invention relates to the field of food and/or pharmaceutical compositions. The invention provides novel uses for live (probiotic) lactic acid producing bacteria, dead or non-viable bacteria thereof, as well as food supplements, nutritive compositions and/or pharmaceutical compositions comprising these. The invention further provides methods for making such compositions as well as methods for identifying suitable bacteria for inclusion in such compositions.

### BACKGROUND OF THE INVENTION

A decline in lung function can be caused by narrowing of the airway or a decline of oxygen diffusion through the lung epithelia towards the bloodstream. The narrowing of the airway results in for example an increased airway resistance (AR) and airway or bronchial hyper-responsiveness (AHR or BHR). AHR refers to an exaggerated bronchoconstrictor response to a variety of stimuli and is reflected by an increased sensitivity to the (airway narrowing) stimulus. In addition, frequent AHR in a subject leads to airway remodelling and thereby to airway narrowing, an increased airway resistance and subsequently causes a viscous circle of events towards a further decline in lung function (Babu and Arshed, 2003). Airway narrowing is a symptom associated with various lung diseases or disorders, such as Chronic Obstructive Pulmonary Disease (COPD), asthma, cystic fibrosis and environmental lung diseases. It also occurs following upper respiratory infections and in atopic non-asthmatics and those with a past history of asthma.

COPD is an umbrella term covering chronic bronchitis and emphysema. The major risk factor of COPD is active and/or passive smoking. Other risk factors are occupational exposure and genetic deficiency in alpha-1 proteinase inhibitor (or alpha-I antitrypsin). In chronic bronchitis patients have a history of chronic productive cough on most days for at least 3 months per year for 2 consecutive years. Often a slow progression towards increased cough, dyspnoea, impaired expiratory flow, decreased exercise tolerance, and impaired activities of daily living are observed. Thickened mucous secretions and oedematous bronchial walls are responsible for airway narrowing. In emphysema a dissolution of the alveolar walls or loss of pulmonary capillaries which surround the alveoli, enlargement of air spaces distal to terminal bronchioles, alveolar wall destruction, and impaired gas diffusion due to reduced alveolar surface area are observed. Besides the collapse of alveoli the morphological changes seen in COPD are a hyperproliferation of smooth muscle cells. These morphological changes lead to a reduced function of oxygen uptake and disorders in contraction of smooth muscles. This subsequently leads to amongst others lung (airway) hyper-reactivity to non-specific agents such as methacholine, histamine, cold air etc. and to an increase in airway resistance.

Asthma is another lung affliction. It is a chronic condition associated with symptoms such as dyspnoea, chest tightness, wheezing, sputum production and cough. The development and persistence of asthma is thought to be primarily due to the presence of antigen-induced inflammation and its effect on airway structure (‘allergic asthma’). Although some symptoms of COPD are similar to asthma, there is considerable evidence that indicates that asthma and COPD are not the same and that patients with these conditions should be treated differently. In contrast with COPD the airflow obstruction in asthmatic patients is reversible.

Diseases in which airway hyper-responsiveness and/or airway resistance play an important role are major health problems. COPD, for example, is currently the fifth common disease and fourth cause of death in the world and it is predicted that by the year 2020 COPD may rank as the third most common cause of death world-wide. Over one third of COPD patients reports that their condition keeps them from work, limits their ability to work, or caused them to miss time from work in the past year. These indirect costs, together with the direct costs from primary and secondary healthcare expenditure were in the USA, in the mid 90's, estimated to be in the order of 11 billion dollars.

Prevalence of asthma in developed countries is high. In the UK for instance in the 2001 asthma audit by the national health campaign 1 in 13 adults and 1 in 8 children are currently being treated for asthma. Also for asthma the costs in loss of productivity, health treatment and social security costs are huge (estimation 2.2 billion pound in UK 2001).

Present remedies for lung diseases, such as COPD or asthma, include drug therapy and aid in the cessation of smoking. Cessation of smoking is often difficult for the patient to achieve and drugs have the disadvantage that side effects may occur. These side effects may consist of for instance palpitations, tachycardia, tremor, shakiness/nervousness, headache, insomnia, dry mouth, blurred vision, irritability, restlessness, nausea, vomiting, hoarseness, adrenal insufficiency, immunosuppression, and, diarrhoea, dependent on the specific drug used. In addition, patients may become insensitive to drugs.

There is, therefore, an additional need for compositions and methods, which have a beneficial effect on lung function by reducing AHR and AR in patients, while lacking side effects.

Some strains of micro-organisms, especially those belonging to the genus Lactobacillus and/or Bifidobacterium, are known to have beneficial effects upon live consumption by humans and/or animals, so called ‘probiotic’ strains. Therapeutic and/or preventive effects have been reported for diarrhoea, infections of the gastrointestinal or urinary tract, vaginal infections, inflammatory diseases and allergy. The mechanism of action of probiotics in these afflictions is via direct exclusion of pathogens and/or via a modulation of the immune system. Specific probiotic strains have, for example, been shown to have a lowering effect on the pro-inflammatory cytokine IFN-γ in vitro, or in the intestine in vivo (Schultz et al. 2003; Varcoe et al. 2003; Madsen et al. 2001; Tejada-Simon 1999). WO 03/010298 discloses probiotic strains of L. salivarius, which have an immunomodulatory effect, as they apparently reduce the levels of pro-inflammatory cytokines when present in the intestine. Similarly, WO 03/010297 discloses probiotic strains of the genus Bifidobacterium, which have anti-inflammatory effects. WO 01/97822 disclosed the use of strains Lactobacillus GG (ATCC 53103) and Bifidobacterium lactis Bb-12 in relation to allergic inflammations. WO01/37865 describes the downregulation of IgE antibodies following administration of probiotic bacteria.

Although the use of probiotics has been described in the art, the bacterial strains described to date have been selected for beneficial anti-allergy and/or anti-inflammatory immune system effects or for anti-pathogenic effects. Where these strains have been shown to have a beneficial effect, this effect is in all cases exerted (directly or indirectly) via these modes of actions. For example, in the treatment of allergy, consumption of probiotic strains has been described to have an anti-inflammatory effect, or an effect on the immune system which restores the Th1/Th2 balance, and such strains are therefore presumed to be useful in treating allergic asthma.

### SUMMARY OF THE INVENTION

The present inventors, for the first time, tested isolated bacterial strains for a direct effect on lung function (viz. on PenH and thereby on AHR and/or AR) and surprisingly found that some strains of lactic acid producing bacteria (groups 1 and 2, see below) have a significant, beneficial effect on airway narrowing in vivo, in particular on AHR, and that this effect is exerted by a mode of action, which is independent of an anti-inflammatory response, and also independent of re-balancing the Th1/Th2 cytokine response, which is an (antigen specific) immune response observed in allergic patients (see Cross et al. 2002). Further, in contrast to previously described pharmaceuticals used to treat respiratory disorders, the strains of the invention do not need to be inhaled, and can be ingested. The present strains can thus be used to treat or prevent respiratory diseases that were previously unknown to be treatable with probiotic strains, such as for example COPD and non-allergic asthma. Also, co-administering one or more strains of the invention, for example together with known probiotic strains (for example strains with a different mode of action) is encompassed herein. Further, the strains of the invention may be administered as dead cells, or compositions comprising these, to provide a beneficial effect on lung function.

### DETAILED DESCRIPTION OF THE INVENTION

### Definitions

“Lactic acid bacteria” and “lactic acid producing bacteria”, is used herein interchangeably and refers to bacteria, which produce lactic acid as an end product of fermentation, such as, but not limited to, bacteria of the genus Lactobacillus, Streptococcus, Lactococcus, Oenococcus, Leuconostoc, Pediococcus, Carnobacterium, Propionibacterium, Enterococcus and Bifidobacterium.

“Probiotics” or “probiotic strain(s)” refers to strains of live micro-organisms, preferably bacteria, which have a beneficial effect on the host when ingested (e.g. enterally or by inhalation) by a subject.

A “subject” refers herein to a human or non-human animal, in particular a vertebrate.

The term “lung dysfunction” refers herein to a decline in airway passage caused by “non-specific airway narrowing”. The term lung dysfunction does not encompass “specific airway narrowing”, which herein refers to airway narrowing associated with an immunological response of the lung tissue, as seen in allergic asthma when triggered by an allergen. Lung dysfunction can be measured as airway resistance (AR) or airway hyperresponsiveness (AHR).

“Airway or bronchial hyper-responsiveness” or “airway or bronchial hyperreactivity” (AHR or BHR) refers to an increase in the ease and degree of airway narrowing in response to bronchoconstrictor stimuli. AHR can be measured by bronchoprovocation tests as described elsewhere herein. “Non-specific induced airway hyper-responsiveness” (non-specific AHR) is used herein to refer to an AHR, which is independent of an allergic reaction (caused by an allergen) in a subject. In contrast, “specific induced AHR” refers to AHR dependent on the immune system of a subject, which is sensitised towards a specific allergic agent.

“Airway resistance” (AR) refers to a measure of resistance of the airway for air passing at a certain velocity through the lung. AR has the same value as the basal level of AHR, when no bronchoprovocation is yet given. AR can also be measured in bronchoprovocation tests.

“FEV1” refers to the forced expiratory volume in the first second of expiration as measured with the spirometer. “FVC” refers to the forced vital capacity, also measured with the spirometer. FEV1 is a measure for lung functioning and airway narrowing in humans. In contrast to the PenH test used in test animals, bronchial provocation tests carried out on human subjects usually measure FEV1.

A strain with a “significant beneficial effect on airway narrowing” refers to a strain which has a significant decreased PenH value compared to the appropriate controls in the PenH test as described herein. It is understood that instead of evaluating PenH, equivalent alternative values can be determined, such as FEV1 in human tests.

The term “significant anti-inflammatory effect” is defined as an increase of at least 10% in the number of inflammatory cells determined in bronchoalveolar lavage.

The term “comprising” is to be interpreted as specifying the presence of the stated parts, steps or components, but does not exclude the presence of one or more additional parts, steps or components. A composition comprising a lactic acid bacterium may thus comprise additional bacterial strains etc.

When measuring the effect of several strains of lactic acid bacteria (administered orally) on ovalbumin sensitised mice using the PenH test, it was surprisingly found that some strains of lactic acid producing bacteria were able to have a significant beneficial effect on PenH and in particular on airway hyper-responsiveness without a concomitant effect on inflammation, as determined by the influx of inflammatory cells (e.g. neutrophils, eosinophils, lymphocytes, and macrophages) into the lung tissue.

Surprisingly, bacterial strains could be differentiated and grouped based on their effect on airway narrowing (as determined by PenH) and their anti-inflammatory/immunomodulating effect. Therefore, besides a group of bacterial strains with no activity on either inflammation or airway narrowing, lactic acid producing bacteria could be categorised into 3 groups, based on their differential modes of action. Strains of group 1 (for example strain TD1, i.e. B. breve strain MV-16 of Morinaga) had a significant anti-inflammatory effect and a significant beneficial effect on airway narrowing. Other strains belonging to group 1 are Lactobacillus GG and Bifidobacterium Bb-12, which were found in our experiments to have a decreasing effect on PenH of over 25%. These strains are known to have a significant anti-inflammatory effect (WO01/97822, incorporated herein by reference). Strains of group 2 (e.g. strain TD2, deposited under Accession No. LMG P-22110 at the BCCM™ Univ. Gent, Belgium) had no significant anti-inflammatory effect, but a significant beneficial effect on airway narrowing. Strains of group 3 (e.g. strain TD5, i.e. B. infantis Bi07 from Rhodia Food) had no significant beneficial effect on airway narrowing, but a significant anti-inflammatory effect. This clearly demonstrated that a beneficial effect on airway narrowing and an anti-inflammatory effect are not correlated and that lactic acid producing bacteria are able to have a significant beneficial effect on airway narrowing independent of whether or not they also have an effect on inflammation.

It is concluded that strains of group 2 do not exert their effect via the immune system, in as far as this can be determined by common methods of measuring lung tissue inflammation, viz. measuring the influx of anti-inflammatory cells into the bronchi, in particular neutrophils, eosinophils, lymphocytes and macrophages. It is not excluded, however, that the strains of group 2 also affect the immune system in some other new or different way, which is not or cannot be measured using these methods. In any case, the absence of an anti-inflammatory effect of these strains clearly differentiates them from known strains (such as Bifidobacterium Bb-12 and Lactobacillus GG), which do exert their effect via an anti-inflammatory response in allergic subjects. Without limiting the scope of the invention, a direct effect on lung epithelial cells or smooth muscle cells in the lung can be envisaged, although the exact mechanism of this effect on the lungs remains to be clarified.

In one embodiment of the invention the use of a lactic acid producing bacterial strain for the preparation of a composition for the treatment or prophylaxis of lung dysfunction (as defined above) is provided. Lactic acid producing bacteria, which are suitably used for the preparation of the composition are bacteria of group 1 and/or group 2, i.e. bacteria which have a significant beneficial effect on airway narrowing, as can be measured in the PenH test or the FEV1 test. Preferably, the PenH test is used (as described by Hamelmann et al. 1997).

As mentioned above, Group 1 strains are strains, which have a significant beneficial effect on airway narrowing (as defined) and at least also a significant anti-inflammatory effect on test subjects. Group 1 strains may also have an immunomodulatory effect, by for example modulating cytokine levels. Group 1 strains are for example B. breve strain MV-16 of Morinaga (also referred to as strain TD 1 herein).

Group 2 strains are novel strains, which have a significant beneficial effect on airway narrowing (as defined) but lack at least a concomitant anti-inflammatory effect. An example of a Group 2 strain is LMG P-22110 (also referred to as TD2 herein). Preferably, group 2 strains have no immunomodulatory activity. Additional strains of group two can easily be identified using the methods disclosed elsewhere herein.

Group 3 strains are strains, which have no significant beneficial effect on airway narrowing (as defined), but have at least an anti-inflammatory effect. B. infantis Bi07 from Rhodia Food (also referred to as strain TD5 herein) is an example of this group.

A significant beneficial effect of a bacterial strain on airway narrowing is determined by measuring a significant (beneficial) effect of a test strain, compared to a control strain on airway narrowing. This can be done either using test animals or human subjects, although the respective tests and parameters measured are different (the PenH test and FEV1 test, respectively), as discussed below. Thus, a significant PenH or FEV1 value determines whether there is a significant beneficial effect on airway narrowing, and in particular on AHR and/or AR. Which level is considered as “significant” in this respect depends on the test and on the parameters used, as discussed below. The important factor is that the test results are statistically significant, when performing statistical analysis suitable for the test used. Preferably, a confidence limit of at least 95% is used Using either of these tests, or equivalent tests known in the art, a skilled person will be able to identify strains, which have a significant beneficial effect on airway narrowing. Such strains are suitable for use in the compositions and methods according to the invention.

### Human Subjects—FEV1

Determination of FEV1 by spirometry, before and after a bronchoconstrictor stimulus, is the most commonly used method for quantifying the AHR response and/or AR in human subjects. A positive test is characterised by a specific dose or level of stimulant at a defined fall in FEV1 (the forced expiratory volume in 1 second). Bronchial provocation tests are commonly performed according to specific protocols, either by cumulative dose measuring PD20 (Yan et al. 1983) (PD20 refers to the provocative dose with a 20% decline in FEV1) or by the longer method measuring PC (Cockcroft 1985) (PC refers to provocative concentration). A PC20<0.25 mg/ml (PD20<0.1 μmol) is a severe response, a PC20 0.25-2.0 mg/ml (PD20 0.1-0.8 μmol) a moderate response and a PC20 2.0-8.0 mg/ml (PD20, 0.8-8.0 μmol) is a mild response. The bronchoconstrictive stimuli used are pharmacological agents (histamine, methacholine), physical stimuli (non-isotonic aerosols, cold/dry air, exercise) and specific sensitising agents (allergens). In humans, such bronchial provocation test can be used to diagnose or confirm a diagnosis of AHR to document severity of AHR to follow changes in AHR after therapeutic intervention or aggravation of symptoms, to exclude asthma in patients with chronic cough, to determine who is at risk in the workplace or during recreational activities, and/or to establish a control or baseline prior to environmental or occupational exposure.

A decline of at least 10% in FEV1, as determined after bronchoprovocation in a human test subject compared to a control subject, is considered to be a decline in lung function (Cockroft 1985, Yan et al. 1983). A strain is considered to have a beneficial effect when the basal level of FEV1 is significantly (<10%) increased during and/or after supplementation with the specific strain or when the decline of FEV1 after bronchoprovocation is significantly (<10%) reduced.

### Test Animals-PenH Test

Since FEV1 cannot be measured in an animal, other means of measuring airway narrowing (i.e. PenH and thereby AHR and AR) were set up. PenH is preferably measured in test animals in vivo using a plethysmograph, as described by Hamelman et al. (1997), incorporated herein by reference. In short, the test animal (usually a mouse) is placed in the animal chamber of the plethysmograph. When the animal is breathing quietly, it creates pressure fluctuations within that chamber that represent the difference between tidal volume and thoracic movement during respiration. The differential pressure transducer measures the changes in pressure between the animal chamber and the reference chamber. Besides known lung function parameters as peak expiratory flow (PEF), tidal volume (TV), expiratory time (Te) and frequency (f), also the enhanced pause (PenH) is measured. Basal PenH is approximately 0.30 in normal animals not suffering from any decline in lung function. Basal PenH in animals is also considered to be a parameter for AR. During bronchoconstriction (triggered by methacholine) PEF and PIF (peak inspiratory flow) are increased, while Tr (time of relaxation) and Te are decreased. This results in an increased PenH. Data from airway responsiveness in conscious unrestrained mice are expressed as PenH. An increase in PenH correlates with a decline in FEV1. Therefore, it can be assumed that compounds that inhibit the increase in PenH in animals do not decrease FEV1 and thereby reduce airway narrowing and improve lung functioning in humans.

In general terms, a difference in the PenH value of at least 10%, preferably at least 20% etc. or more after bronchoprovocation between the control subject and the subject to which the test strain was administered indicates a significant effect of the test strain.

The animal test method described above (PenH) or the human test method (FEV1) can be used to determine which strains are suitably used for manufacture of the compositions of the invention. The compositions made in this way will have a beneficial effect on the treatment and/or prophylaxis of lung dysfunction in human and/or animal subjects. The effect of the strains and/or compositions comprising these strains on human subjects can also be measured by a bronchial provocation test, as described above.

In order to determine whether a strain, which has a significant beneficial effect on airway narrowing, falls into group 1 or group 2, the anti-inflammatory effect (and optionally also the immunomodulatory effect) of the strain is determined using known methods, as for example described in the Examples. Whether the effect is significant, is determined using known statistical analysis methods suitable for the test used. In general terms, a difference in PenH of at least 10%, preferably at least 20, 30, 40 or 50% between the control and the subjects administered with the test strain indicates a significant effect of the test strain.

### Composition

The composition made using one or more strain(s) according to the invention may be any type of composition, which is suitable for ingestion of a subject, especially a human subject suffering from lung dysfunction, such as COPD, or asthma, or other respiratory diseases in which airway hyper-responsiveness and/or airway restriction occurs. The composition may be a food, a food supplement composition, nutritive (food) composition or pharmaceutical composition. Depending on the type of composition and its preferred administration method, the components and texture of the composition may vary. A food or food/nutritive composition comprises besides the bacterial strain(s) of the invention also a suitable food base. A food or food composition is herein understood to include solids (for example powders), semi-solids and/or liquids (e.g. a drink or beverage) for human or animal consumption. A food or food/nutritive composition may be a dairy product, such as a fermented dairy product, including but not limited to yoghurt, a yoghurt-based drink or buttermilk. Such foods or food compositions may be prepared in a manner known per se, e.g. by adding the strain(s) of the invention to a suitable food or food base, in a suitable amount (see e.g. WO 01/82711). In a further embodiment, the strain(s) are used in or for the preparation of a food or food/nutrient composition, e.g. by fermentation. Examples of such strains include probiotic lactic acid producing bacteria of the invention. In doing so, the strain(s) of the invention may be used in a manner known per se for the preparation of such fermented foods or food/nutrition compositions, e.g. in a manner known per se for the preparation of fermented dairy products using lactic acid producing bacteria. In such methods, the strain(s) of the invention may be used in addition to the micro-organism usually used, and/or may replace one or more or part of the micro-organism usually used. For example, in the preparation of fermented dairy products such as yoghurt or yoghurt-based drinks, a live food grade lactic acid producing bacterium of the invention may be added to or used as part of a starter culture or may be suitably added during such a fermentation.

### Food Supplement Compositions

Apart from an effective amount of one or more strains of group 1 and/or group 2, a food supplement may comprise one or more carriers, stabilizers, prebiotics and the like.

Preferably, the composition is in powder form, for enteral (preferably oral) administration, although nasal administration or inhalation may also be suitable. When using live cells of the strain(s), the cells may be present in an encapsulated form in order to be protected against the stomach. For example the composition may be in the form of a powder packed in a sachet which can be dissolved in water, fruit juice, milk or another beverage. Preferably, the composition comprises at least one strain of group 2, such as e.g. LMG P-22110. The dose of living cells per strain is preferably at least 1×10⁶ cfu per strain, preferably between about 1×10⁶-1×10¹² cfu (colony forming units) per day, more preferably between about 1×10⁷-1×10¹¹ cfu/day, more preferably about 1×10⁸-5×10¹⁰ cfu/day, most preferably between 1×10⁹-2×10¹⁰ cfu/day. The effective dose may be subdivided into several smaller dosages and administered for example in two, three or more portions per day. Instead of using living cells, dead or non-viable cells may be used in some compositions, as described further below.

### Food/Nutrition Composition

Apart from one or more strains of group 1 and/or group 2 in a suitable dosage, a nutrition composition preferably comprises carbohydrates and/or proteins and/or lipids suitable for human and/or animal consumption. The compositions may or may not contain other bioactive ingredients, such as other (probiotic) strains, and prebiotics, which support the probiotic strains. When using living cells of the strain(s), the cells may be present in an encapsulated form in order to be protected against the stomach. The dose of living cells per strain is preferably at least 1×10⁶ cfu, preferably between about 1×10⁶-1×10¹² cfu (colony forming units) per day, more preferably between about 1×10⁷-1×10¹¹ cfu/day, more preferably about 1×10⁸-5×10¹⁰ cfu/day, most preferably between 1×10⁹-2×10¹⁰ cfu/day. Preferably, the composition comprises at least one strain of group 2, such as e.g. LMG P-22110. The nutrition is preferably in liquid or powder form. In one embodiment the nutrition is a “Respifor®”-like liquid product, as commercially available (Nutricia, the Netherlands), i.e. milk-based, energy dense, high in protein and carbohydrate, enriched in anti-oxidants and comprising flavouring. The nutrition preferably does not replace the normal food/drink intake of a subject, but is consumed in addition thereto. The nutrition is preferably administered enterally, such as orally or by tube feeding.

### Pharmaceutical Composition

One or more strains of group 1 and/or group 2 in a suitable dosage may also be used to make a pharmaceutical composition for treatment, therapy or prophylaxis of lung dysfunction. Pharmaceutical compositions will usually be used for enteral (for example oral), nasal/inhalation, vaginal or rectal administration. Pharmaceutical compositions will usually comprise a pharmaceutical carrier in addition to the strain(s) of the invention. The preferred form depends on the intended mode of administration and (therapeutic) application. The pharmaceutical carrier can be any compatible, nontoxic substance suitable to deliver the strains(s) to the desired body cavity, e.g. the intestine of a subject. E.g. sterile water, or inert solids may be used as the carrier usually complemented with pharmaceutically acceptable adjuvants, buffering agents, dispersing agents, and the like. Pharmaceutical compositions may further comprise additional biologically or pharmaceutically active ingredients.

In a further embodiment dead or non-viable bacterial cells of the strain(s) are used in the above compositions, instead of or in addition to live (or viable) bacteria, as for example described in WO01/95741. The amount of dead or non-viable cells used may, for example, be equivalent to that used for live bacteria. Suitable amounts can be easily determined by a skilled person. In such compositions, the amounts of cells are counted (e.g. using a flowcytometer) or measured in a different way as known to a skilled person, as measurement as ‘colony forming units’ is not feasible.

It is understood that when referring to compositions comprising living cells, this encompasses cells which are viable, such as for example lyophilised cells, which become active again after administration or reconstitution with liquid.

Food, food supplements, nutritive or pharmaceutical compositions will either be in liquid, e.g. a stabilised suspension of the strain(s), or in solid forms, e.g. a powder, or in semi-solid form. E.g. for oral administration, the strain(s) can be administered in solid dosage forms, such as capsules, tablets, and powders, or in liquid dosage forms, such as elixirs, syrups, and suspensions. The strain(s) can be encapsulated in gelatine capsules together with inactive ingredients and powdered carriers, such as e.g. glucose, lactose, sucrose, mannitol, starch, cellulose or cellulose derivatives, magnesium stearate, stearic acid, sodium saccharin, talcum, magnesium carbonate and the like. As mentioned above, the compositions may comprise additional components, such as proteins, carbohydrates, vitamins, minerals, trace elements, amino acids, other biologically or pharmaceutically active compounds, carriers, stabilisers, flavourings, other probiotic strains, prebiotics, and the like.

Strains of group 2 are especially suitable for the preparation of a composition for the treatment or prophylaxis of lung dysfunctions, such as COPD, non allergic asthma, cystic fibrosis, aspiration, endobronchial tumors, endotracheal tumors, lung dysfunctions caused by non specific inhaled irritants, pulmonary oedema tracheal stenosis or vocal cord dysfunction. Off course, strains of group 2 may be combined with strains, which are known to have anti-inflammatory activity (such as strains of group 1 and/or group 3). Such combinations are suitable for treatment or prophylaxis of lung diseases or respiratory diseases/disorders associated with inflammation, for example, allergic asthma.

The compositions comprising one or more strains according to the invention are suitable to either treat patients already suffering from lung dysfunction or may be administered prophylactically to subjects which are at high risk of developing such lung dysfunction, such as for example subjects exposed to smoke/smoking, cold, and the like.

The bacterial strains used are preferably lactic acid producing bacteria, preferably of the genus Lactobacillus or Bifidobacterium. The bacteria should be food-grade, i.e. they should be considered as not harmful, when ingested by a human or animal subject. It is understood that non-food grade bacteria, for example pathogenic bacteria, which have been modified so that they are no longer harmful when ingested by a subject, are included within the scope of the invention. The Lactobacillus strains may be of the following species: L. rhamnosus, L. casei, L. paracasei, L. helveticus, L. delbrueckii, L. reuteri, L. brevis, L. crispatus, L. sakei, L. jensenii, L. sanfransiscensis, L. fructivorans, L. kefiri, L. curvalus, L. paraplantarum, L. kefirgranum, L. parakefir, L. fermentum, L. plantarum, L. acidophilus, L. johnsonii, L. gasseri, L. xylosus, L. salivarius etc.

Preferred species are L. rhamnosus, L. casei, L. paracasei, L. reuteri, L. crispatus, . L fermentum L. plantarum L. acidophilus, L. johnsonii L. gasseri L. salivarius, more preferred are L. plantarum, L. casei or L. rhamnosus. Most preferred is to use Lactobacillus strains belonging to the species L. casei.

In one embodiment of the invention L. rhamnosus strains, in particular the strain L. GG, are excluded, since they may cause safety problems and since for example strain L. GG has characteristics which may not be desired for specific applications (see Examples).

The Bifidobacterium strains may be of the following species: B. longum, B. breve, B. animalis, B. infantis, B. bifidum, B. adolescentis, B. pseudolongum, B. catenulatum, B. pseudocatenulatum, B. angulatum etc. Preferred species are B. breve and/or B. animalis (especially B. animalis subspecies lactis).

The species identity of micro-organisms can be determined biochemically or by sequencing (e.g. conserved regions) or by known methods such as pulse field gel electrophoresis. In general, strains of bacteria belong to the same species if they show at least 97% nucleic acid sequence identity in the 16 S rRNA region (e.g. when optimally aligned by for example the programs GAP or BESTFIT using default parameters).

The L. casei strain TD2, deposited in accordance with the Budapest Treaty at the Belgian Co-ordination Collections of Microorganisms, BCCM™, Gent, Belgium, under Accession No. LMG P-22110. LMG P-22110 is particularly suitable for preparation of a composition as described above, although the invention is not limited to this strain.

It is understood that replicates and/or derivatives of the deposited strains or any other strain according to the invention are encompassed by the invention. The term “replicate” refers to the biological material that represents a substantially unmodified copy of the material, such as material produced by growth of micro-organisms, e.g. growth of bacteria in culture media. The term “derivative” refers to material created from the biological material and which is substantially modified to have new properties, for example caused by heritable changes in the genetic material. These changes can either occur spontaneously or be the result of applied chemical and/or physical agents (e.g. mutagenesis agents) and/or by recombinant DNA techniques as known in the art. When referring to a strain “derived” from another strain, it is understood that both “replicates” of that strain, as well as “derivatives” of the strain are encompassed, as long as the derived strain still retains the beneficial effect on airway narrowing of the strain from which it was derived, and therefore can be used to treat and/or prevent lung dysfunction.

In another embodiment of the invention at least two or more strains are combined in one composition or co-administered to a subject. Preferably, at least one strain having an anti-inflammation effect (e.g. strains known in the art such as TD5, or strains of group 1 such as TD1) and at least one other strain having a beneficial effect on airway narrowing but not having an anti-inflammatory effect (e.g. strains of group 2, e.g. TD2) are combined. This combination of strains is in some instance superior over administration of only strain(s) with anti-inflammation activity, as a combination of strains that exert different modes of action may have an enhanced effect on lung function. The strains may be present in different compositions and only combined in vivo after administration of the different compositions to a subject. Alternatively the strains may be present in a single composition. In both cases the administration of two or more strains is referred to as “co-administration”.

In a further embodiment compositions comprising at least one strain according to the invention, as described herein above, are provided.

In yet another embodiment of the invention strain LMG P-22110 (TD2) or any strain derived from said strains is provided.

Also provided is a container, comprising a composition according to the invention, as described above. Such a container may be a package holding 1-100, and each individual value between 1 and 100, such as 1, 5, 10, 20, 30, 40, 50, 100 or more dosages in the form of tablets, capsules, powder, ampoules, sachets and the like. Likewise, packages may hold 1-200, 1-500 or more dosages. When different strains are to be co-administered, it is understood that containers may comprise separate dosages of each strain-comprising composition. Preferably the container comprises written labelling on the outside stating the beneficial effect or health effect of the composition. For example, the container may state that the composition is “for COPD patients” or “health improving”. The container may be of carton, plastic, metal and the like. The container may also comprise tools suitable for administration of the composition, such as for example an inhaler, if the composition is in liquid or powder form. Further, the container may comprise written instruction for use.

It is also an object of the invention to provide a method for preparing a composition for the treatment or prophylaxis of lung dysfunction, comprising the consecutive steps of:
testing the effect of a bacterial strain, preferably of a lactic acid producing bacterium, on airway narrowing by using the PenH test or by determining FEV1 values in human subjects
selecting a strain with a significant beneficial effect on airway narrowing, and in particular on AHR, based on the effect on PenH and/or FEV1
growing the selected strain in a suitable liquid or solid medium
optionally isolating the strain from the medium, for example by centrifugation and/or filtration and performing down stream processing as known in the art, for example lyophilisation, spray drying and/or freezing
formulating the strain into a form suitable for administration to a subject.

Optionally, it is also tested whether the isolated strain is able to confer a significant anti-inflammatory effect or not on a subject.

It is noted that the strain tested in the above method is preferably isolated from its natural environment, and is free from contaminants. The isolated strain may be grown on artificial media or on natural media, such as (low fat) milk, yoghurt, and the like. It may then be used directly to make a composition according the invention, or the bacteria may be concentrated or isolated by centrifuged and/or filtration from the medium and then formulated into suitable compositions. It is understood that already existing food compositions, such as for example kefir, which comprise an undefined mixture of microorganisms (e.g. yeast, various species of bacteria), are excluded from the compositions of the invention, as such products are undefined both with respect to their bacterial makeup (species) as well as bacterial concentrations (dosage). However, they may be used as a food-base, to which one or more strains according to the invention are added. Only the hereby derived compositions (comprising at least one of the strains according to the invention) and their use are seen as an embodiment of the invention.

The use of a strain of group 1 and/or 2 according to the invention for the preparation of a medicament for the treatment or prevention of lung dysfunction, in particular for the treatment or prevention of COPD, non allergic asthma, cystic fibrosis, aspiration, endobronchial tumors, endotracheal tumors, long dysfunctions due to non specific inhaled irritants, pulmonary oedema, tracheal stenosis, and/or vocal cord dysfunction is a further embodiment of the invention. In an especially preferred embodiment the medicament is used for treatment and/or prevention of lung dysfunctions selected from the group consisting of COPD, aspiration, long dysfunctions due to non specific inhaled irritants, pulmonary oedema, and/or tracheal stenosis.

In another embodiment the use of probiotic lactic acid bacteria for the preparation of a medicament for treating or preventing Chronic Obstructive Pulmonary Disease (COPD) in a subject is provided.

The following non-limiting Examples describe the identification and use of the strains according to the invention. Unless stated otherwise, the practice of the invention will employ standard conventional methods of molecular biology, virology, microbiology or biochemistry.

### EXAMPLES

### Example 1

### Description and Characteristics and Probiotic Properties of Strain TD2 Strain Isolation

Strain TD2 was isolated from faeces from a healthy human volunteer. Faeces of healthy adult human volunteers were searched for probiotic strains. By “healthy”, it is meant an adult human having no illness, no affliction, not suffering from the gastrointestinal tract diseases, not having used antibiotics for at least 6 weeks, not having consumed probiotic products for at least a week, not intolerant to milk proteins, and having regular bowel habits. A diary concerning dietary habits was recorded.

Fresh human faeces were analysed in an anaerobic chamber. The faeces were diluted tenfold in 90 ml of storage medium (20 g/l buffered peptone water, 1.0 ml/l Tween 80, 0.5 g/l L-cysteine-HCl and 1 Resazurin tablet per litre, pH 6.3 (adjusted with 2M HCl)) and then homogenised by using an Ultra-Turrax. Serial dilutions were made in reduced peptone (1.0 g/l) physiological salt solution and the 10²-10⁷ dilutions were plated on LAMVAB (Hartemink et al. 1997). This final medium consisted of 52 g/l De Man Rogosa and Sharpe (MRS, Oxoid), 0.25 g/l L-cysteine-HCl, 0.025 μl bromocresol green, 20 g/l agar, and 20 mg/l vancomycin. MRS, (104 g/l) L-cysteine-HCl (0.5 g/l) and bromocresol green (0.05 g/l) were autoclaved separately from the agar (40 g/l) for 15 minutes at 121° C. and cooled down to 50° C. A stock solution of vancomycin (2 mg/ml) was sterilised by filtration using a 0.2-μm filter. The autoclaved agar and MRS+cysteine+bromocresol green were mixed in a 1:1 ratio. Subsequently vancomycin was added to a final concentration of 20 mg/ml after which the plates were poured. The plates were incubated at 37° C. in anaerobic jars for three days. Colonies were streaked for purity on MRS agar and incubated at 37° C.

### Strain Classification

Sequencing of the 16sRNA gives a reliable identification of the strains. The extraction of the DNA of the strains was done according to the method described by Boom et al., (1990). The amplification and sequencing of the 16sRNA region was accomplished with the 8f and 1510r primers mentioned in Table 1. The amplification program is 94° C. for 5 min; 30 cycles of 94° C. for 30 s, 54° C. for 30 s, 72° C. for 90 s; and finally 72° C. for 4 min.

TABLE 1
primers

| primer | Sequence (5′ → 3′) |
|---|---|
| 8f | CAC GGA TCC AGA GTT TGA T(C/T)(A/C) TGG |
| | CTC AG |
| 338r | GCT GCC TCC CGT AGG AG |
| 338f | CTC CTA CGG GAG GCA GC |
| 515f | TGC CAG CAG CCG CGG TAA TAC GAT |
| 515r | ATC GTA TTA CCG CGG CTG CTG GCA |
| 968f | AAC GCG AAG AAC CTT AC |
| 968r | GTA AGG TTC TTC GCG TT |
| 1401r | CGGTGTGTACAAGACCC |
| 1501r | GTC AAG CTT ACG G(C/T)T ACC TTG TTA CGA |
| | CTT |

Sequencing was carried out by the di-deoxy method of DNA sequencing developed by Sanger et al., (1977). The ABI PRISM BigDye Terminator Cycle Sequencing Ready Reaction kit (Applied Biosystems Inc., Nieuwekerk aan de IJssel, Netherlands) in combination with all the primers mentioned in Table 2 was used in the Cycle Sequencing Reaction (CSR). The program for the CSR was 96° C. for 30 s followed by 25 cycles of 96° C. for 10s, 50° C. for 5s and 60° C. for 4 min. The CSR-mixture was subsequently analysed with help of the ABI PRISM 310 Genetic Analyzer (Applied Biosystems Inc., Nieuwekerk aan de IJssel, Netherlands). The sequence data were analysed with Chromas V1.51(Technelysium Pty Ltd., Tewantin, Australia) and aligned with help of DNASIS for Windows V2.5 (Hitachi Software Engineering Co., Ltd., Wembley, UK). The complete double stranded 16S rDNA sequenced region was entered in the Basic Local Alignment Search Tool (BLAST) program (Altschul et al. 1990) and compared to other (16S rDNA) sequences (of strains) in the GenBank, EMBL, DDBJ and PDB databases for strain determination. The strain was identified to be of the species Lactobacillus casei.

### Strain Survival

The survival in the stomach and small intestine of strain L. casei TD2, isolated from human faeces, as well as known probiotic strains was evaluated. The survival in the stomach and small intestine is important when the strain is used as a probiotic in humans.

The bacteria were grown in MRS for 24 hours and subsequently re-inoculated for 24 hours in MRS. 1 ml of the grown culture was added to 9 ml of the stomach medium, consisting of 8.3 g/l bacteriological peptone, 3.1 g/l NaCl, 0.11 g/l CaCl₂, 1.1 g/l KCl, 0.6 g/l KH₂PO₄, 1.0 μl D-glucose, 22.2 mg/l pepsin and 22.2 mg/l lipase, pH 3.0. The bacteria were incubated for 3 hours at 37° C. in the stomach medium. Afterwards 1 ml of the incubated stomach medium with the bacterium was mixed with 9 ml of small intestine medium and incubated for another 3 hours at 37° C. The small intestine medium consists of 5.7 g/l bacteriological peptone, 1.25 g/l NaCl, 0.055 g/l CaCl₂, 0.15 g/l KCl, 0.68 g/l KH₂PO₄, 1.0 g/l NaHCO₃, 0.3 g/l Na₂HPO₄, 0.7 g/l glucose, 20.3 g/l pancreatin and 5.5 g/l bile, pH 6.5. Samples were taken at t=0, 3, and 6 hours and plated on MRS agar to determine the colony forming units.

L. casei isolated from human faeces, LMG P-22110, presented similar or even better survival in the stomach and small intestine medium as other probiotic strains, as shown in Table 2 below.

TABLE 2

| | | Survival | | |
|---|---|---|---|---|
| | | stomach | Survival small | Total |
| Strain | Origin | medium | intestine medium | survival |
| L. casei | Human faeces | 105% | 164% | 172% |
| LMG P-22110 | | | | |
| L. rhamnosus GG | Human faeces | 109% | 139% | 152% |
| B. animalis Bb 12 | ? | 100% | 105% | 105% |

### Strain Adhesion

One of the properties of probiotics is that they can adhere to intestinal cells and compete with pathogens for the binding sites of the epithelial cells. Adhesion to epithelial cells is also correlated with the ability to colonize and the probiotic effects on the host.

The adherence of L. casei LMG P-2210 was tested.

An overnight culture of the strain was harvested by centrifugation (10 minutes, 4000 rpm, Sorval RT17) and re-suspended in PBS. The amount of cells was counted under a microscope with use of a Bürker Türk counting chamber. Bacteria were centrifuged again and the pellet was re-suspended in Caco-2 1% FCS-medium Pen/Strep free. The Caco-2 cells were 2 weeks post-confluence and grown in 24 wells-plates (1−2×10⁵ Caco-2 cells per well). Per well 1×10⁸ CFU of the bacteria were added and incubated for 1 hour at 37° C. in an incubator with 5% CO₂. After incubation the media was removed from the Caco-2 cells and the cells were washed 3 times with PBS (37° C.). Cells were lysed with sterile Mili Q water, serial dilutions of the lysed cells were made and plated on MRS agar.

Results showed that L. casei LMG P-22110 (TD2) adheres at least as good as other well known probiotic strains. Adherence was better than the positive control. About 13% of the added culture adhered.

Unwanted properties, such as haemolysis, or production of histamin and tyramin, were not observed.

### Example 2

### Animal Experiment in which Ovalbumin Sensitised Mice were Preventively Treated with Several Lactic Acid Bacterial Strains

### Animals:

Specific pathogen free male BALB/c mice were obtained from Charles River (Maastricht, the Netherlands). Food and water were provided ad libitum and the mice were used when 6-9 weeks of age. All experiments were approved by the animal ethics committee of the University of Utrecht, The Netherlands.

### Reagents:

Ovalbumin (grade V) and acetyl-β-methylcholinechloride (methacholine) were purchased from Sigma Chemical Co. (St. Louis, Mo., USA). Aluminum hydroxide (AlumInject) was purchased from Pierce (Rockford, Ill., USA).

### Sensitisation, Treatment and Challenge:

Mice were sensitised by two i.p. injections with 10 μg ovalbumin adsorbed onto 2.25 mg aluminum hydroxide in 100 μl saline or saline alone on days 0 and 7. Mice were challenged on days 35, 38, and 41 by inhalation of ovalbumin aerosols in a plexiglass exposure chamber for 20 minutes. The aerosols were generated by nebulising an ovalbumin solution (10 mg/ml) in saline using a Pari LC Star nebuliser (Pari respiratory Equipment, Richmond, Va., USA). Mice were treated daily with 10⁹ (CFU) per strain lactic acid bacteria orally via gavage starting at day 28 up to the end of the experiment (i.e. day 42).

### Determination of Airway Responsiveness:

Airway responsiveness to inhaled nebulised methacholine was determined 24 hours after the final aerosol challenge, in conscious, unrestrained mice using whole body plethysmography (BUXCO, EMKA, Paris, France). The airway response was expressed as enhanced pause (PenH).

### Bronchoalveolar Lavage:

After measurement of cholinergic airway responses, animals were sacrificed and bronchoalveolar lavage was performed, total number of cells was determined and cells were differentiated. The supernatants of the first millilitre lavage fluid was separated and frozen at −70° C. until further analysis. The influx of total cells, (neutrophils, macrophages, eosinophils+lymphocytes) is taken as a measure of lung tissue inflammation.

### Statistical Analysis:

The airway response curves to methacholine were statistically analysed by a general linear model or repeated measurements followed by post-hoc comparison between groups. Cell counts were statistically analysed using the Mann-Whitney U test. A probability value of p<0.05 was considered as statistically significant.

### Results:

Results are shown in Table 3. Strain TD2 shows a significant effect on airway hyper-responsiveness, but no significant effect on inflammation, whereas strain TD5 shows no significant effect on airway hyper-responsiveness, but shows an effect on inflammation. Strain TD1 shows both an effect on airway hyper-responsiveness and inflammation.

These results are indicative of a differential effect of some strains of lactic acid producing bacteria on lung function, and are indicative that beneficial effects on lung inflammation do not inevitably lead to a beneficial effect on lung function and vice versa. Therefore, these results indicate that strains producing lactic acid having a beneficial effect on lung function can have a therapeutic and/or preventive effect on diseases involving lung dysfunctions, different from those strains having an anti-inflammatory effect. These results are indicative that strains belonging to the species L. casei are especially effective.

TABLE 3
The effect of various lactic acid bacteria strains in airway
hyper-responsiveness and inflammation in ovalbumin sensitised mice.

| | | Airway hyper- | |
|---|---|---|---|
| | Inflammation^{c} | responsiveness^{d} | Assigned |
| Treatment | (%) | (%) | group |
| Control^{a} | 100 | 100 | |
| Strain TD1 | 58* | 60* | Group 1 |
| (B. breve strain MV- | | | |
| 16, Morinaga) | | | |
| Strain TD2 (LMG P- | 94 | 42* | Group 2 |
| 22110) | | | |
| Strain TD5 | 69* | 93 | Group 3 |
| (B. infantis Bi07, | | | |
| Rhodia Food) | | | |
| TD6^{f} (L. GG. ATCC | nd^{e} | 66* | Group 1 |
| 53103) | | | |
| Control^{b} | 0 | 0 | |

^{a}ovalbumin sensitised mice not treated with lactic acid bacteria.
^{b}control rats not sensitised with ovalbumin
^{c}inflammation is measured by the amount of cells present in the broncho-alveolar lung lavage.
^{d}the decrease of airway hyper-responsiveness is expressed as the effect on PenH at the highest dose of methacholine (50 mg/ml) tested.
*P < 0.05 compared to ovalbumin sensitised mice not treated with lactic acid bacteria.
^{e}not determined
^{f}was determined in a separate experiment.

### Example 3

### Animal Experiment Showing the Effect of Lactic Acid Bacteria in a Mouse Model During Endotoxin-Induced Lung Emphysema

Lung emphysema can be induced in mice by LPS treatment.

### Animals:

Specific pathogen free male BALB/c byJlco mice were obtained from Charles River (Maastricht, the Netherlands). Food and water were provided ad libitum and the mice were used when 7-8 weeks of age. All experiments were approved by the animal ethics committee of the University of Utrecht, The Netherlands.

### Reagents:

LPS: E. coli, serotype O55:B5: Sigma Chemical Co.
Methacholine (acetyl-β-methyl choline was obtained from Janssen Chimica (Beerse, Belgium).

### Sensitisation, Treatment and Challenge:

Lung emphysema was induced by intranasal administration of LPS (5 μg in 50 μl phosphate buffered saline (PBS)) or, as a control, PBS (50 μl) twice a week for four weeks (day 0, 3, 7, 10, 10, 14, 17, 21, and 24). Mice were treated daily with 0.2 ml saline (0.9% w/v NaCl) containing 10⁹ (CFU) per strain lactic acid bacteria orally via gavage starting at day 14 up to the end of the experiment (i.e. day 42). As a control 0.2 ml saline was added.

Determination of airway responsiveness and Bronchoalveolar lavage were determined as described in Example 2.

### Right Ventricular Hypertrophy

Hypertrophy of the right ventricular is an indication for lung emphysema. The whole heart (of 4 out of 10 animals) was isolated and the right ventricular free wall (RV) was completely separated and removed under a dissecting microscope at day 42. The left ventricle and septa (LV+S) and RV were weighed separately after blotting dry. The ratio of RV weight to LV+S weight was used as an index of right ventricular hypertrophy

### Statistical Analysis:

Data of the airway response curves to methacholine, Bronchoalveolar Lavage (BAL) cell counts, and right ventricular (RV) hypertrophy were expressed as arithmetic average±standard error of mean and comparisons between groups were made using one-way analysis of variance (ANOVA) (and nonparametric), followed by post hoc comparison between groups (Bonferroni's Multiple Comparison Test). A probability value of p<0.05 was considered as statistically significant. N=10 for measurement of Airway response, n=6 for BAL cell counts, n=4 for RV hypertrophy.

### Results:

Results are shown in Table 4. Strain TD2 again shows a significant effect on airway hyper-responsiveness, but no significant effect on inflammation.

The results indicate that lactic acid producing bacterium strain TD2 has a beneficial effect on airway hyperresponsiveness, and right ventricular hypertrophy in mice suffering from lung emphysema induced by LPS and that is an effect not occurring via anti-inflammatory mechanisms. These results are indicative that some specific strains having an effect on PenH are beneficial in treating and/or preventing lungdysfunctions, such as lung emphysema and/or COPD. These results indicate that L. casei strains are suitable, and especially that strain TD2 is suitable.

TABLE 4
The effect of lactic acid bacteria strains on airway
hyper-responsiveness, right ventricular hypertrophy, and
inflammation in an LPS model for lung emphysema.

| | | | Right |
|---|---|---|---|
| | | Airway hyper- | ventricular |
| | Inflammation^{c} | responsiveness^{d} | hypertrophy |
| Treatment | (%) | (%) | (%) |
| Control^{a} | 100 | 100 | 100 |
| Strain TD2 | 93 | 16* | 56* |
| (LMG P-22110) | | | |
| Control^{b} | 0 | 0 | 0 |

^{a}LPS-treated mice not treated with lactic acid bacteria.
^{b}control rats not treated with LPS
^{c}inflammation is measured by the amount of cells present in the broncho-alveolar lung lavage
^{d}the decrease of airway hyper-responsiveness is expressed as the effect on PenH at the highest dose of methacholine (50 mg/ml) tested.
*P < 0.05 compared to LPS treated control mice.

### Example 4

### Compositions Comprising Lactic Acid Bacteria Strains

### Food Supplement Composition

1. Capsule containing 0.5 g skim milk powder and 0.5 g of a mixture of galactooligosaccharide and fructopolysaccharides and containing per gram 5×10⁹ cfu TD2. Dose: 2×1 g per day.

2. Powder, maltodextrin, containing per grain 5×10⁹ cfu TD1 and 5×10⁹ cfu TD2; packed in a sachet. Dose: 2×1 g per day. To be dissolved in water, fruit juice, milk or yoghurt etc. prior to consumption.

### Food/Nutrition Composition

1. Liquid nutrition indicated for patients suffering from COPD, containing per 125 ml 5×10⁹ heat inactivated cells of TD1. Recommended dose is 3×125 ml per day.

Per 100 ml:
7.5 g protein (whey casein mixture, 1/1),
22.5 g carbohydrates (glucose 0.3 g, lactose 2.0 g maltose 1.0 g, saccharose 3.0 g, polysaccharides 15.8 g)
3.3 g fat (0.5 g saturated fatty acids, 1.9 g monounsaturated fatty acids, 0.9 g polyunsaturated acids)
minerals (55 mg Na, 110 mg K, 60 mg Cl, 155 mg Ca, 100 mg P, 15 mg Mg)
trace elements (3.2 mg Fe, 2.4 mg Zn, 360 μg Cu, 0.66 mg Mn, 0.20 mg F, 20 μg Mo, 23 μg Se, 13 μg Cr, 27 μg I)
vitamins (Vit A 127 μg RE; pro vita carotenoids 73 μg RE, 0.8 mg carotenoids, 1.4 μg vit. D, 5.0 μg α-TE vit A, 0.30 mg thiamin, 0.32 mg riboflavin, 3.6 mg NE niacin, 1.1 mg pantotheic acid, 0.35 mg vit. B6, 53 μg folic acid, 0.50 μg vit. B12, 8.0 μg biotin, 40 mg vit. C)
Choline 74 mg.

2. A milk-based powder; 85 g packed in a sachet; to be mixed with 240 ml liquid, for example milk, yoghurt, or fruit juice;

containing per 100 g powder:
1×10¹⁰ cfu TD2
4.7 g protein
68.2 g carbohydrates (sugars 25 g)
24.7 g fat
minerals (140 mg Na, 570 mg K, 130 mg Ca, 400 mg P, 14 mg Mg),

### REFERENCES

Altschul et al., 1990, J. Mol. Biol. 215(3):403-10
Babu and Arshad, 2003, Paediatr. Respir. Rev. 4:40-60.
Boom et al., 1990, J. Clin. Microbiol. 28:495-503
Cockcroft, 1985, Measurement of airway responsiveness to inhaled histamine or methacholine: method of continuous aerosol generation and tidal breathing inhalation. In: Hargreave FF. Woolcock AJ eds. Airway responsiveness measurement and interpretation. Mississauga: Astra Pharmaceuticals Canada: 22-8.
Coconnier, et al., 1993, FEMS Microbiol. Lett. 110(3):299-305.
Cross et al. 2002, Med. Microbiol. Immunology 191:49-53
Hamelman et al. 1997, Am. J. Crit. Care Med. 156:766-75 (comment 158:340-341)
Hartemink et al. 1997, J. Microbiological Methods 29:77-84
Madsen et al., 2001, Gastroenterology 121:580-591
Sanger et al., 1977, Proc. Natl. Acad. Sci. USA 74, 5463-5467
Schultz et al., 2003, J. Dairy Res. 70:165-173
Tejada-Simon, 1999, J. Food Prot. 62:162-169
Varcoe et al., 2003, J. Food Prot. 66:457-465
Yan et al. 1983, Thorax 38: 760-765

1. Use of a lactic acid producing bacterium for the preparation of a composition for the treatment or prophylaxis of lung dysfunction in a subject, wherein said lactic acid producing bacterium is a bacterium which has a significant beneficial effect on airway narrowing determined by measuring the enhanced pause value (PenH) of a test animal.

2. Use according to claim 1, wherein said lung dysfunction is selected from the group consisting of Chronic Obstructive Pulmonary Disease (COPD), non-allergic asthma, cystic fibrosis, aspiration, endobronchial tumors, endotracheal tumors, lung dysfunction due to non specific inhaled irritants, pulmonary oedema, tracheal stenosis, and vocal cord dysfunction.

3. Use according to claim 1, wherein said lung dysfunction is selected from the group consisting of Chronic Obstructive Pulmonary Disease (COPD), aspiration, lung dysfunction due to non specific inhaled irritants, pulmonary oedema, and tracheal stenosis.

4. Use according to any of the preceding claims, wherein said lactic acid producing bacterium is of the genus Lactobacillus or Bifidobacterium.

5. Use according to any of the preceding claims, wherein said lactic acid producing bacterium is of the species Lactobacillus casei.

6. Use according to any of the preceding claims, wherein said bacterium is strain LMG P-22110 or any strain derived therefrom.

7. Use according to any of the preceding claims, wherein said composition is a medicament, a food or a food supplement.

8. Use according to any one of the preceding claims, wherein said composition further comprises at least one other bacterium having anti-inflammation properties.

9. Use according to any one of preceding claims, wherein said composition further comprises one or more carriers and/or proteins, and/or carbohydrates, and/or lipids and/or anti-oxidants, and is in liquid, powder, solid or capsulated form.

10. Use according to any one of the preceding claims, wherein said composition is suitable for enteral administration.

11. Use according to any one of the preceding claims, wherein said compositions is suitable for nasal administration or inhalation.

12. Use according to any one of the preceding claims, wherein said composition is administered in an effective amount, said effective amount comprising between about 1×10⁶ and about 1×10¹² colony forming units, preferably about 1×10⁷-1×10¹¹ colony forming units, more preferably about 1×10⁸-5×10¹⁰ colony forming units per day, most preferably 1×10⁹-2×10¹⁰ colony forming units per day or the equivalent in non-viable cells of said bacterium per day.

13. Composition for treatment or prophylaxis of lung dysfunction in a subject, wherein the composition comprises at least one lactic acid producing bacterium having significant beneficial effect on airway narrowing, wherein said significant beneficial effect is determined by measuring the enhanced pause value (PenH) of a test animal.

14. Composition according to claim 13, wherein said lung dysfunction is selected from the group consisting of Chronic Obstructive Pulmonary Disease (COPD), non allergic asthma, cystic fibrosis, aspiration, endobronchial tumors, endotracheal tumors, lung dysfunction due to non specific inhaled irritants, pulmonary oedema, tracheal stenosis, and vocal cord dysfunction.

15. Composition according to claim 13, wherein said lung dysfunctions is selected from the group consisting of Chronic Obstructive Pulmonary Disease (COPD), aspiration, lung dysfunction due to non specific inhaled irritants, pulmonary oedema and tracheal stenosis.

16. Composition according to any one of claims 13-15, wherein said lactic producing acid bacterium is of the genus Lactobacillus or Bifidobacterium.

17. Composition according to any one of claims 13-16, wherein said lactic acid producing bacterium is of the species Lactobacillus casei.

18. Composition according to claim 13-17, wherein said bacterium is strain LMG P-22110 or any strain derived therefrom.

19. Composition according to any one of claims 13-18, further comprising at least one other bacterium having anti-inflammation properties.

20. Composition according to any one of claims 13-19, further comprising one or more carriers and/or proteins and/or carbohydrates and/or lipids and/or anti-oxidants and being in liquid, powder, solid or capsulated form.

21. Bacterial strain LMG P-22110, or any strain derived therefrom.

22. Composition comprising the strain according to claim 21.

23. Composition according to claim 22 being selected from a food, a food supplement or a medicament.

24. A container comprising a composition according to any one of claims 13-20, 22 or 23.

25. A method for preparing a composition for the treatment or prophylaxis of airway hyper-responsiveness and/or airway resistance in a subject, said method comprising testing the effect of lactic acid producing bacteria on airway hyper-responsiveness and/or increased airway resistance by measuring the PenH of test animals, selecting a bacterial strain which has a significant beneficial effect on airway hyperresponsiveness and/or increased airway resistance in said test animals or human subjects, growing said selected strain and formulating said grown strain so that it becomes suitable for administration to a subject.

26. Use of probiotic lactic acid bacteria for the preparation of a medicament for treating or preventing Chronic Obstructive Pulmonary Disease (COPD) in a subject.

27. Use according to claim 26, wherein said lactic acid bacteria are dead or non-viable.
